(19) United States
(12) Patent Application Publication
(10) Pub. No.: US 2019/0054251 A1
(43) Pub. Date: Feb. 21, 2019

(54) MEDICINE DELIVERY DEVICE

(71) Applicant: QUALCOMM Incorporated, San Diego, CA (US)

(72) Inventors: James PIERONEK, San Diego, CA (US); Robert GANTON, San Diego, CA (US); Robert BALLAM, Eatons Hill (AU); John Earl AMSCHLER, Del Mar, CA (US); Paul Robert Hoffman, San Diego, CA (US)

(21) Appl. No.: 16/104,057

(22) Filed: Aug. 16, 2018

Related U.S. Application Data
(60) Provisional application No. 62/547,100, filed on Aug. 17, 2017.

(51) Int. Cl. A61M 5/315

(52) U.S. Cl. CPC A61M 5/31568; A61M 5/31528; A61M 5/31511; A61M 5/31535; A61M 5/31501; A61M 5/31556; A61M 2005/3126; A61M 2205/3317; A61M 2205/3553; A61M 2205/3569; A61M 2205/50; A61M 2205/3306; A61M 5/31585

(57) ABSTRACT

Methods, systems, computer-readable media, and apparatuses for facilitating administering of medicine are disclosed.

### RELATED APPLICATION

This patent application claims priority to U.S. Provisional Patent Application Ser. No. 62/547,100, filed Aug. 17, 2017, entitled “A DOSAGE SENSING MEDICINE DELIVERY DEVICE” which is assigned to the assignee hereof and is incorporated herein by reference in its entirety for all purposes.

### BACKGROUND

Aspects of the disclosure relate to medicine delivering devices, and more particularly to techniques for sensing a dosage setting at a medicine delivery device.

Certain medical devices may be used to deliver a medicine to a user. An example medical device is an injection device (e.g., a syringe, an injection pen, etc.). The injection device holds the medicine in fluid form (e.g., liquid, gas, etc.), and includes a variable dosage setting mechanism (e.g., a piston) which allows a user to set a dosage of the medicine to be dispensed. The user may estimate the dosage on by reading the numerical scale markings on the injection device. After setting the dosage, the user may then operate the injection device to inject the dosage of the medicine into the user's body. The actual dosage set by the user may deviate from the dosage intended by the user, which in turn affects the proper administering of the medicine.

### SUMMARY

In some embodiments, a medicine delivery device is provided. The medicine delivery device comprises a container configured to store a medicine, a movable component coupled with the container and configured to set a dosage of the medicine to be dispensed, a dispensing mechanism coupled with the container and configured to deliver the dosage of the medicine; and one or more sensors configured to generate data samples related to a movement of the movable component. The medicine delivery device further comprises a processing circuit coupled with the one or more sensors and configured to: determine, based on the data samples, a direction of at least one movement and a distance of the at least one movement; determine, based on the direction of the at least one movement and the distance of the at least one movement, a dosage of the medicine set by the movable component; compare the dosage against a pre-set threshold; and perform one or more actions related to delivery of the dosage of the medicine from the container based on a result of the comparison.

In some aspects, the movable component includes one or more detection targets for the one or more sensors. The data samples comprises an indication of absence and an indication of presence of a detection target of the one or more detection targets.

In some aspects, the one or more sensors comprise at least two switches. The one or more detection targets comprise one or more structures capable of causing the at least two switches to enter an on-state or an off-state. The indication of absence of a detection target of the one or more detection targets comprises an indication that one of the at least two switches is in the off-state. The indication of presence of a detection target of the one or more detection targets comprises an indication that the one of the at least two switches is in the on-state.

In some aspects, the at least two switches are mechanical switches; and the one or more structures are capable of causing the at least two switches to enter the on-state by applying a force to the at least two switches.

In some aspects, the at least two switches are transistor switches. The one or more structures store charges and are capable of causing the at least two switches to enter the on-state by applying at least some of the charges to gates of the transistor switches.

In some aspects, the one or more sensors comprise one or more optical sensors. The one or more detection targets comprise one or more markers.

In some aspects, the one or more detection targets include a resistive path between two contacts. The one or more sensors are coupled with the two contacts and are configured to generate data samples of a resistance measurement of the resistive path. The processing circuit is configured to determine the direction of at least one movement and the distance of the at least one movement based on a change in the resistance measurement reflected in the data samples.

In some aspects, the resistive path has an non-uniform distribution of resistance.

In some aspects, the processing circuit is configured to: determine a counter value based on the data samples; determine a net displacement of the movable component based on the counter value; and determine the dosage based on the net displacement.

In some aspects, the processing circuit is configured to: determine, based on the data samples, whether the counter value is to be updated; and based on a determination that the counter value is to be updated, increment or decrement the counter value based on whether the direction of the at least one movement is of a first direction or of a second direction.

In some aspects, the determination of whether the counter value is to be updated comprises the processing circuit being configured to: determine whether there is a change in the indication of absence or in the indication of presence of a detection target of the one or more detection targets between a current data sample and a prior data sample obtained by one of the one or more sensors; and update the counter value based on the change.

In some aspects, the one or more sensors comprises a first sensor and a second sensor; wherein the data samples comprise: first data samples including a first sequence of indications of absence and indications of presence of a detection target obtained by the first sensor; and second data samples including a second sequence of indications of absence and indications of presence of the detection target obtained by the second sensor. The processing circuit is configured to determine the direction of the at least one movement based on the first data samples and the second data samples.

In some aspects, the at least one movement comprises a linear movement and a rotational movement. The one or more sensors comprises: a first set of one or more sensors configured to generate first data associated with the linear movement, and a second set of one or more sensors configured to generate second data including an angular displacement of the rotational movement. The processing circuit is configured to: determine a combined distance of the linear movement based on a combination of the first data and the second data; and determine the dosage based on the combined distance.

In some aspects, the movable component comprises a piston coupled with a threaded shaft. The first data is associated with a pre-determined scale for measuring the linear movement of the piston. The angular displacement included in the second data corresponds to a fraction of the pre-determined scale for measuring the rotational movement of the threaded shaft.

In some aspects, the medicine delivery device further comprises a wireless interface coupled with the processing circuit. The processing circuit is further configured to perform the one or more actions comprising: transmitting information related to the delivery of the dosage of the medicine via the wireless interface to a client device associated with a user, to cause the client device to display the information; transmitting the information for displaying at a display interface of the medicine delivery device; or any combination thereof.

In some aspects, the information comprise a notification of insufficient dosage of medicine left for dispensing. In some aspects, the information comprise an indication about the dosage. In some aspects, the information is transmitted to a drug adherence or compliance system for enforcing one or more pre-determined drug adherence or compliance rules.

In some aspects, the processing circuit is further configured to perform the one or more actions comprising: based on a determination that the dosage is not equal to the pre-set threshold, controlling the dispensing mechanism not to dispense the medicine, wherein the pre-set threshold comprises one of: a pre-set maximum amount, or a pre-set dosage.

In some aspects, the processing circuit is further configured to perform the one or more actions comprising: based on a determination that the dosage is not equal to the pre-set threshold, adjusting the dosage using the movable component such that the dosage becomes equal to the pre-set threshold.

In some embodiments, a method is provided. The method comprises: receiving, from a sensor, data samples related to a movement of a movable component relative to a container that stores a medicine, the sensor, the movable component and the container being part of a medicine delivery device; determining, based on the data samples, a direction of at least one movement of the movable component and a distance of the at least one movement; determining, based on the direction of the at least one movement and the distance of the at least one movement, a dosage of the medicine set by the movable component; comparing the dosage against a pre-set threshold; and controlling, based on a result of the comparison, the medicine delivery device to perform one or more actions related to delivery of the dosage of the medicine from the container.

In some aspects, the movable component includes one or more detection targets for the one or more sensors. The data samples comprises an indication of absence and an indication of presence of a detection target of the one or more detection targets.

In some aspects, the one or more sensors comprise at least two switches. The one or more detection targets comprise one or more structures capable of causing the at least two switches to enter an on-state or an off-state. The indication of absence of a detection target of the one or more detection targets comprises an indication that one of the at least two switches is in the off-state. The indication of presence of a detection target of the one or more detection targets comprises an indication that the one of the at least two switches is in the on-state.

In some aspects, the at least two switches are mechanical switches; the one or more structures are capable of causing the at least two switches to enter the on-state by applying a force to the at least two switches.

In some aspects, the at least two switches are transistor switches; and the one or more structures store charges and are capable of causing the at least two switches to enter the on-state by applying at least some of the charges to gates of the transistor switches.

In some aspects, the one or more sensors comprise one or more optical sensors; and the one or more detection targets comprise one or more markers.

In some aspects, the one or more detection targets include a resistive path between two contacts. The one or more sensors are coupled with the two contacts and are configured to generate data samples of a resistance measurement of the resistive path. The method further comprises determining the direction of at least one movement and the distance of the at least one movement based on a change in the resistance measurement reflected in the data samples.

In some aspects, the resistive path has an non-uniform distribution of resistance.

In some aspects, the method further comprises: determining a counter value based on the data samples; determining a net displacement of the movable component based on the counter value; and determining the dosage based on the net displacement.

In some aspects, the method further comprises: determining, based on the data samples, whether the counter value is to be updated; and based on a determination that the counter value is to be updated, incrementing or decrementing the counter value based on whether the direction of the at least one movement is of a first direction or of a second direction.

In some aspects, determining whether the counter value is to be updated comprises: determining whether there is a change in the indication of absence or in the indication of presence of a detection target of the one or more detection targets between a current data sample and a prior data sample obtained by one of the one or more sensors; and updating the counter value based on the change.

In some aspects, the one or more sensors comprises a first sensor and a second sensor; wherein the data samples comprise: first data samples including a first sequence of indications of absence and indications of presence of a detection target obtained by the first sensor; and second data samples including a second sequence of indications of absence and indications of presence of the detection target obtained by the second sensor. The method further comprises: determining the direction of the at least one movement based on the first data samples and the second data samples.

In some aspects, the at least one movement comprises a linear movement and a rotational movement. The one or more sensors comprises: a first set of one or more sensors configured to generate first data associated with the linear movement, and a second set of one or more sensors configured to generate second data including an angular displacement of the rotational movement. The method further comprises: determining a combined distance of the linear movement based on a combination of the first data and the second data; and determining the dosage based on the combined distance.

In some aspects, the movable component comprises a piston coupled with a threaded shaft. The first data is associated with a pre-determined scale for measuring the linear movement of the piston. The angular displacement included in the second data corresponds to a fraction of the pre-determined scale for measuring the rotational movement of the threaded shaft.

In some aspects, performing the one or more actions comprises: transmitting information related to the delivery of the dosage of the medicine via the wireless interface to a client device associated with a user, to cause the client device to display the information, transmitting the information for displaying at a display interface of the medicine delivery device, or any combination thereof. In some aspects, the information comprise a notification of insufficient dosage of medicine left for dispensing. In some aspects, the information comprise an indication about the dosage. In some aspects, the information is transmitted to a drug adherence or compliance system for enforcing one or more pre-determined drug adherence or compliance rules.

In some aspects, performing the one or more actions comprises: based on determining that the dosage is not equal to the pre-set threshold, controlling the dispensing mechanism not to dispense the medicine, wherein the pre-set threshold comprises one of: a pre-set maximum amount, or a pre-set dosage.

In some aspects, performing the one or more actions comprising: based on determining that the dosage is not equal to the pre-set threshold, adjusting the dosage using the movable component such that the dosage becomes equal to the pre-set threshold.

In some embodiments, a non-transitory computer readable medium is provided. The non-transitory computer readable medium stores a set of instructions that, when executed by a hardware processor, causes the hardware processor to perform: receiving, from a sensor, data samples related to a movement of a movable component relative to a container that stores a medicine, the sensor, the movable component and the container being part of a medicine delivery device; determining, based on the data samples, a direction of at least one movement of the movable component and a distance of the at least one movement; determining, based on the direction of the at least one movement and the distance of the at least one movement, a dosage of the medicine set by the movable component; comparing the dosage against a pre-set threshold; and controlling, based on a result of the comparison, the medicine delivery device to perform one or more actions related to delivery of the dosage of the medicine from the container.

In some embodiments, an apparatus is provided. The apparatus comprises: means for storing a medicine; means for setting a dosage of the medicine to be dispensed; means for delivering the dosage of the medicine; means for generating data samples related to a movement of the means for setting the dosage; means for determining, based on the data samples, a direction of at least one movement and a distance of the means for setting the dosage; means for determining, based on the direction of the at least one movement and the distance of the at least one movement, a dosage of the medicine set by the means for setting the dosage; means for comparing the dosage against a pre-set threshold; and means for performing one or more actions related to delivery of the dosage of the medicine from the means for storing the medicine based on a result of the comparison.

### BRIEF DESCRIPTION OF DRAWINGS

Non-limiting and non-exhaustive aspects are described with reference to the following figures, wherein like reference numerals refer to like parts throughout the various figures unless otherwise specified.

Aspects of the disclosure are illustrated by way of example. In the accompanying figures, like reference numbers indicate similar elements.

FIG. 1 illustrates a simplified diagram of a system for providing information about the administration of medicine by a medicine delivery device;

FIGS. 2A and 2B illustrate a simplified diagram of an example medicine delivery device, according to certain aspects of the present disclosure;

FIG. 3 illustrates a simplified block diagram of example components of medicine delivery device of FIG. 2A and FIG. 2B;

FIG. 4 illustrates a simplified diagram of the internal structure of medicine delivery device of FIG. 2A and FIG. 2B;

FIGS. 5A-5B illustrate example components of a medicine delivery device for dosage estimation, according to certain aspects of the present disclosure;

FIGS. 6A-6C illustrate example operations of the medicine delivery device of FIGS. 5A-5B, according to certain aspects of the present disclosure;

FIG. 7 illustrates example components of a medicine delivery device for dosage estimation, according to certain aspects of the present disclosure;

FIGS. 8A-8C illustrate example components a medicine delivery device for dosage estimation, according to certain aspects of the present disclosure; and

FIG. 9 illustrates an example of a method for facilitate administering of medicine, according to certain aspects of the present disclosure.

### DETAILED DESCRIPTION

Several illustrative examples will now be described with respect to the accompanying drawings, which form a part hereof. While particular examples, in which one or more aspects of the disclosure may be implemented, are described below, other examples may be used and various modifications may be made without departing from the scope of the disclosure or the spirit of the appended claims.

A medicine delivery device, such as an injection device, enables administering of a pre-set dosage of medicine to a user. The medicine delivery may hold the medicine in liquid form, and includes a variable dosage setting mechanism (e.g., a piston, dosage ring, limiting mechanism to set piston movement limits, etc.) which allows a user to set a dosage of the medicine to be dispensed. The user may estimate the dosage by reading the numerical scale markings on the injection device. After setting the dosage, the user may operate the injection device to inject the dosage of the medicine into the user's body.

Although an injection device with a variable dosage setting mechanism provides the flexibility in in setting a dosage, such a device may also introduce dosage error. The dosage error may represent a difference between the actual dosage administered by the injection device and the dosage intended by the user. The dosage error may be caused by, for example, human error, as well as the accuracy limitation of human operation of the dosage setting mechanism. For example, the user may have read the wrong numerical scale markings, interpreted the numerical scale markings incorrectly, etc., and unwittingly set a wrong dosage. As another example, the user may operate the dosage setting mechanism based correct reading and/or interpretation of the numerical scale markings correctly, but the user may only adjust the dosage to a certain precision and cannot completely eliminate dosage error. In both cases, the user may not be informed about the actual dosage of the medicine administered. The dosage error may have adverse effects on the user. The adverse effects may be further exacerbated when the user is not aware of the dosage error.

Disclosed are techniques to improve the accuracy of dosage setting, which may facilitate administering of correct dosage of medicine to a user. In some embodiments, a medicine delivery device includes a container configured to store medicine, a movable component coupled with the container and configured to set a dosage of the medicine to be dispensed, a dosage dispensing mechanism to dispense the dosage of the medicine, and one or more sensors configured to generate data samples related to a movement of the movable component. The medicine delivery device also includes a processing circuit coupled with the one or more sensors. The processing circuit is configured to determine, based on the data samples, a direction of at least one movement and a distance of the at least one movement and, based on the direction of the at least one movement and the distance of the at least one movement, determine a dosage of the medicine set by the movable component. The processing circuit may compare the dosage against a pre-set threshold, and perform one more actions based on a result of the comparison.

In some embodiments, the processing circuit may be configured to compare the dosage against the pre-set threshold to detect dosage error, and the one or more actions may include adjusting the movable component to correct for the dosage error. In some embodiments, the processing circuit may be configured to compare the dosage against the pre-set threshold to detect potential over-dosage, and the one or more actions may include, for example, disabling the dosage dispensing mechanism to prevent the dosage from being dispensed. In some embodiments, the processing circuit may transmit information about the dosage set by the movable component (before or after the adjustment) to a client device to cause the client device to display the information. The client device may be associated with the user, and the information may be transmitted to prompt the user to correct the dosage. The client device may also be part of a drug adherence or compliance system, and the transmitted information may be for enforcing one or more pre-determined drug adherence or compliance rules.

Various techniques are disclosed to estimate a net distance of movement of the movable component to estimate the dosage set by the movable component. In some embodiments, the movable component may include a shaft and a piston. The shaft may move linearly within the container to set a position of the piston, which may define a volume of a space within the container for holding the dosage. The shaft may include a set of discrete detection targets to be sensed by the one or more sensors. As the shaft moves with respect to the one or more sensors, at least some of the set of detection targets also moves across the one or more sensors, and the one or more sensors may detect a pattern of absence and presence of a detection target of the set of detection targets. A counter may be used to track a number of presence (or absence) of the detection target. Moreover, the counter may be incremented when the piston moves in a first direction associated with an increase of dosage, and may be decremented when the piston moves in a second direction associated with a decrease of dosage. Based on the net counter value after the counting-ups and counting-downs, the counter may provide tracking of a net distance of movement of the piston, as well as the direction of the movement, to determine the actual dosage. Various techniques, such as through mechanical contact, optical sensing, capacitive sensing, etc., may be employed to sense the discrete detection targets, to generate triggering events for updating the counter.

In some embodiments, the net distance of movement by the movable component may also be estimated by measuring a physical quantity that changes with a position of the shaft with respect to the one or more sensors. For example, the shaft may provide a resistive path with non-uniform unit resistance. Two electric contact points coupled with two of the one or more sensors may be in contact with two different points on the resistive path. As the shaft moves, due to the non-uniform unit resistance, the resistance between the two electric contact points may change. The change in the resistance may be used to estimate a direction as well as a distance of movement of the shaft.

The disclosed techniques may improve the accuracy of dosage setting as well as safety of medicine delivery. For example, with the disclosed techniques, the medicine delivery device does not rely entirely on the user's manual operation of the dosage setting mechanism to set the dosage. Instead, the medicine delivery device may perform an estimation of the dosage by tracking a direction and a distance of movement of the movable component, and validate the user's operation based on the estimation. As an example, the processing circuit may be preprogrammed to deliver a discrete set of dosages (e.g., in the steps of 0.5 mL, 1 mL, 1.5 mL, etc.). The processing circuit may check the actual dosage set by the movable component under the manual operation of the user and find the closest programmed dosage, and deliver that programmed dosage instead of the dosage set by the user. As another example, the processing circuit may check the actual dosage set against a threshold indicating over-dosage, and may disable the dosage delivery mechanism to prevent over-dosage. All these may reduce the likelihood of delivery an incorrect dosage due to, for example, human operation error (e.g., misreading or misinterpreting the numerical scale markings), insufficient precision of human operation, intentional over-dosage, etc. All these may facilitate administering of correct dosage of medicine to a user and safe use of medicine.

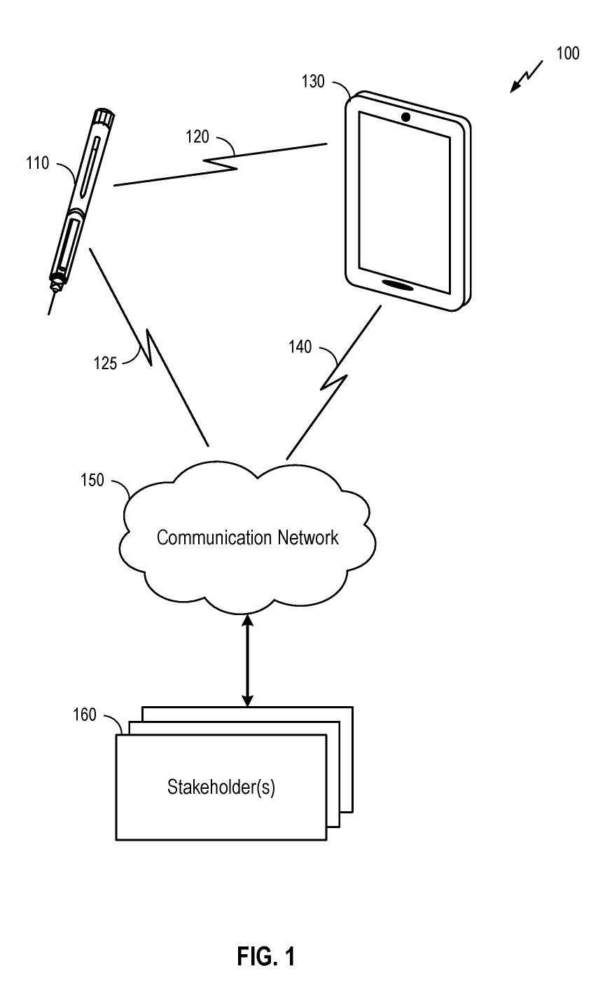

FIG. 1 is a system 100 for providing information about the administration of medicine by a medicine delivery device 110 to one or more stakeholder(s) 160. Here, the system 100 may comprise medicine delivery device 110 as described herein, along with a connecting device 130, communication network 150, and the stakeholder(s) 160. Medicine delivery device 100 may be an device configured to delivery medicine in liquid form. It will be understood, however, that embodiments of system 100 may include a different configuration of components, the addition and/or omission of various components, and/or the like, depending on desired functionality. Moreover, it will be understood that techniques described herein may be utilized in a medicine delivery device 110 that may not necessarily be part of a larger system, such as the system 100 illustrated in FIG. 1.

Medicine delivery device 110, which is described in more detail herein below, is used to administer a medicine to a patient. In of FIG. 1, medicine delivery device 110 may be an injection device such as, for example, a syringe, an injection pen, etc. Here, a person (e.g., a doctor, nurse, or patient him/herself) may administer the medicine by engaging a physical mechanism (e.g., pressing down on a plunger, actuating automatic injection, etc.). Through the physical mechanism, a dose of the medicine may be injected into the patient's skin via a needle of medicine delivery device 110 inserted into the patient's skin. In some embodiments, once the medicine is administered, medicine delivery device 110 may then register, store and transmit data associated with the administration of the medicine to connecting device 130. This data may be transmitted wirelessly via a wireless communication link 120, using any of a variety of wireless technologies as described in further detail below. That said, some embodiments may additionally or alternatively utilize wired communication.

Connecting device 130 may comprise any of a variety of electronic devices capable of receiving information from medicine delivery device 110 and communicating information to the stakeholder(s) 160 via communication network 150. This may include, for example, a mobile phone, tablet, laptop, portable media player, personal computer, or similar device. In some embodiments, connecting device 130 may comprise a specialized device utilized for purposes of conveying information from medicine delivery device 110 (and possibly other medical devices) to stakeholder(s) 160. In some embodiments, the connecting device 130 may comprise a device owned and operated by the patient (e.g., the patient's mobile phone). In other embodiments, the connecting device 130 may be owned and/or operated by another entity, such as a healthcare provider, insurance company, government agency, etc.

Connecting device 130 may execute an application to provide the data processing and/or relaying functionality illustrated in FIG. 1. In some embodiments, the application may be configurable by a user, or may simply be downloaded to connecting device 130 and executed automatically. The application may help establish communication link 120 between medicine delivery device 110 and the connecting device 130, which may or may not require input from the user, depending on desired functionality. In some embodiments, the application may provide instructions to a user on proper use of medicine delivery device 110 and/or feedback to a user about the detected use of medicine delivery device 110. As to be discussed in more details below, medicine delivery device 110 may detect a dosage set by the patient, and transmit information related to the detected dosage to connecting device 130 (e.g., whether incorrect dosage is set), as part of the feedback. Additional and/or alternative functionality of an application executed by the connecting device 130 may be utilized as desired such as, for example, relaying of the data to a remote destination, interacting with the patient about the medicine administration, etc.

Communication network 150 may comprise any of a variety of data communication networks, depending on desired functionality. Communication network 150 may include any combination of radio frequency (RF), optical fiber, satellite, and/or other wireless and/or wired communication technologies. In some embodiments, communication network 150 may comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (Cat-M) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

Communication link 140 between connecting device 130 and communication network 150 may vary, depending on the technologies utilized by these components of the system 100. For examples where connecting device 130 is a smart phone capable of connecting with a cellular network and/or a Wi-Fi® network, communication link 140 may comprise a wireless communication link utilizing the mobile phone's cellular or Wi-Fi® functionality. In embodiments where connecting device 130 is a personal computer, communication link 140 may comprise a wired communication link that accesses communication network 150 via a cable or digital subscriber line (DSL) modem.

It may be noted that some embodiments may not utilize a connecting device 130 to relay data to the communication network 150. In such embodiments, medicine delivery device 110 may connect directly to communication network 150 (as shown in FIG. 1 by communication link 125, which may be used in addition to or as an alternative to communication link 120). For example, medicine delivery device 110 may comprise a Long Term Evolution (LTE) category M (Cat-M) device, NarrowBand IoT (NB-IoT), or other Low Power Wide Area Network (LPWAN). Additionally or alternatively, medicine delivery device 110 may comprise wireless technology similar to the corresponding functionality of connecting device 130 described above. In such embodiments, the communication network may additionally or alternatively comprise a Bluetooth Mesh network (such as CSRMesh), a Wi-Fi network, Zigbee, or WWAN (such as LTE, including Cat-M, or 5G). In some embodiments, medicine delivery device 110 may connect both with the communication network 150 via communication link 125 and with connecting device 130. In such embodiments, connecting device 130 may not need to separately communicate information regarding medicine delivery device 110 to stakeholders 160, but instead the medicine delivery device 110 may communicate this information directly to the stakeholders 160 via the communication network 150.

As noted above, the stakeholder(s) 160 may include any of a variety of entities with an interest in the proper administration of medicine by medicine delivery device 110. This may include an individual practitioner (e.g., a doctor or nurse), a hospital, a drug manufacturer, an insurance provider (or other payer), a government agency or other health organization, and/or the like. In some embodiments, the user of medicine delivery device 110 (e.g., the patient) may also be a stakeholder 160 to which information regarding the use of medicine delivery device 110 is provided. Governmental health regulations and/or legal agreements between the patient and/or stakeholder(s) 160 may apply to the dissemination of information regarding the administration of a drug by medicine delivery device 110 to stakeholder(s) 160. Here, as mentioned above and described in further detail below, medicine delivery device 110 may detect a dosage set by the patient, and transmit information related to the detected dosage to stakeholder(s) 160 (e.g., the patient operating connecting device 130, or other people via other devices).

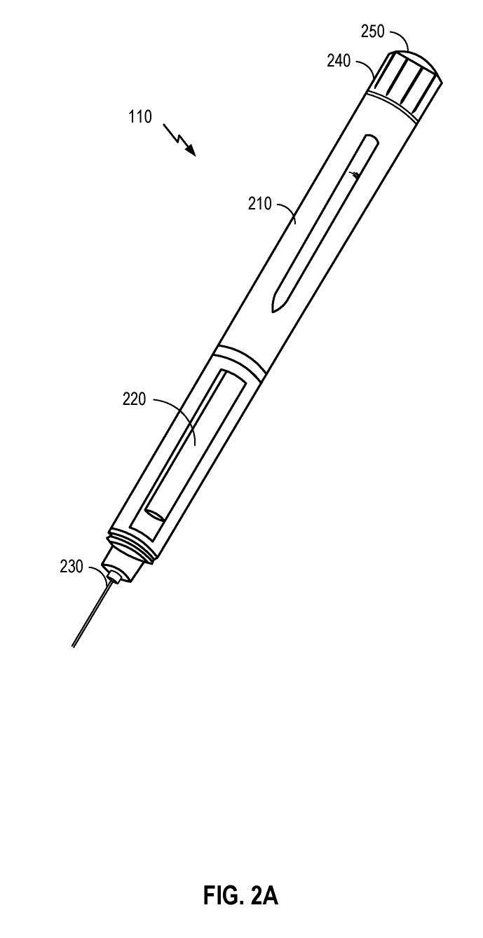

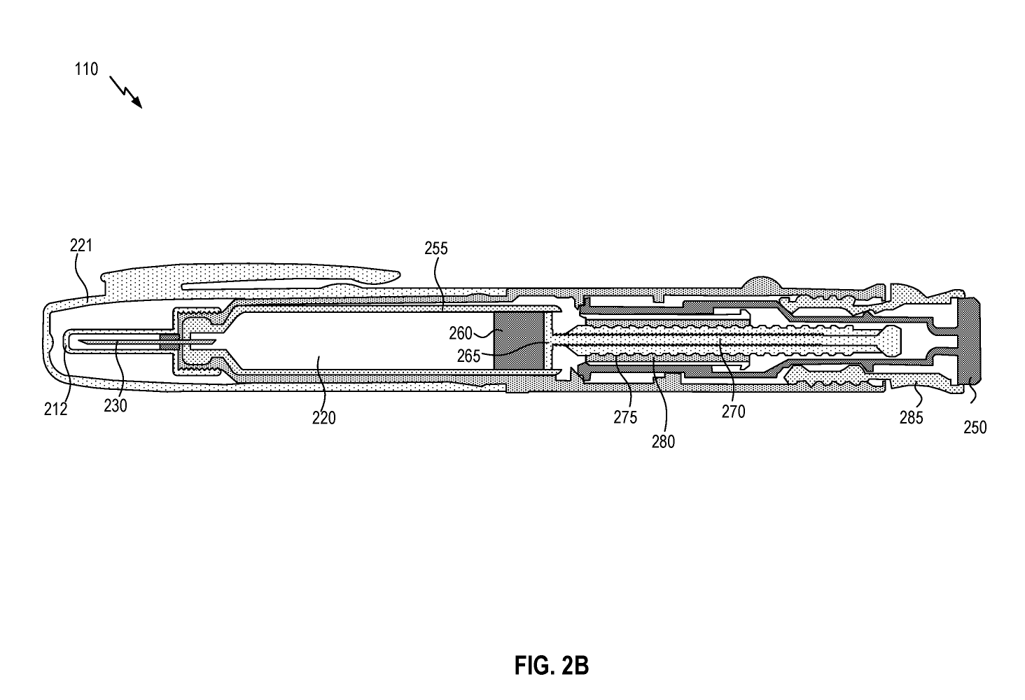

FIG. 2A is an illustration of a medicine delivery device 110, according to certain aspects of the present disclosure. Here, a body 210 of medicine delivery device 110 may house dose dispensing and dose control mechanisms, including electrical and mechanical components, for setting a dosage of medicine to be administered. Mechanical components of a dose dispensing mechanism may include a movable component (e.g., a piston) controlled by the dose control mechanism and configured to displace a volume of the medicine through the reservoir chamber 220 and out of needle assembly 230. Embodiments of medicine delivery device 110 further includes a dose knob 240 that may be adjusted (e.g., by turning the knob clockwise or counterclockwise) to alter the dosage to be administered by medicine delivery device 110. The dosage may be administered by pressing dose dispensing button 250, which may be coupled to a dose dispensing mechanism to control the dispensing of the drug.

FIG. 2B is a cross-sectional view of medicine delivery device 110, according to an embodiment. In the illustration in FIG. 2B, some components not illustrated in FIG. 2A are shown, including the pen cap 221 and inner needle cap 212. The pen injector further includes a cartridge 255 that stores the medicine and comprises reservoir chamber 220. (In some embodiments, the cartridge may be replaceable, enabling medicine delivery device 110 to be used with multiple cartridges.) During the administration of the drug, a piston 260 is pushed by head 265 of a drive stem 270, displacing the drug in reservoir chamber 220 to administer the drug. Drive stem 270 may be screw driven, having threads 275 that feed drive stem 270 through a nut 280. When the user presses dose dispensing button 250, the medicine in reservoir chamber 220 may be displaced via movement of drive stem 270 and piston 260. A volume of space within reservoir chamber 220 for holding the medicine to be displaced may be regulated by dose selector 285.

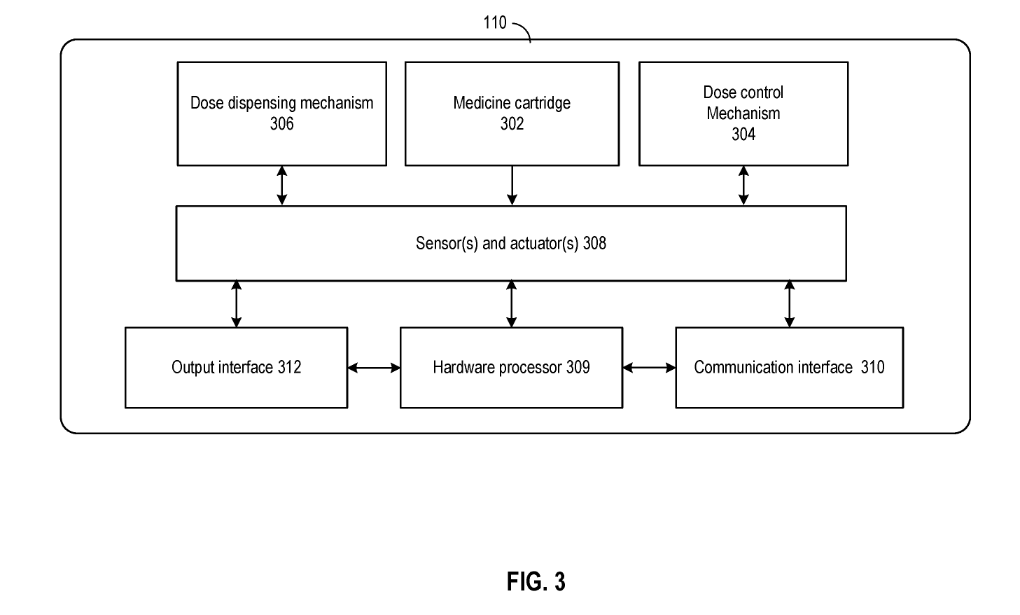

It will be understood, however, that medicine delivery device 110 illustrated in FIG. 2A and FIG. 2B is provided as a non-limiting example, according to an example. Alternative examples may vary in size, shape, and/or other ways. A medicine delivery device 110 may be described more generally as having various components as illustrated in FIG. 3.

FIG. 3 is a block diagram illustrating example components of medicine delivery device 110, according to certain aspects of the present disclosure. Medicine delivery device 110 may include a housing (not shown) structured to hold a medicine cartridge 302, which may store medicine to be dispensed by medicine delivery device 110. Medicine delivery device 110 may also include a dose control mechanism 304 to select or set a dosage of the medicine to be dispensed. For example, dose control mechanism 304 may include a piston shaft or knob to set a volume of medicine held within medicine cartridge to be administered. Medicine delivery device 110 further includes a dose dispensing mechanism 306 to dispense a dose of the drug, from medicine cartridge 302, based on the dosage selected or set by dose control mechanism 304.

Medicine delivery device 110 may include other devices to facilitate administering of medicine. In the example of FIG. 3, medicine delivery device 110 includes sensor(s) and actuator(s) 308, and a hardware processor 309. Sensor(s) and actuator(s) 308 may include sensors and actuators to control the operations of the actuators based on the information collected by the sensor. For example, the sensors of sensor(s) and actuator(s) 308 may collect information of certain physical conditions at, for example, medicine cartridge 302, dose control mechanism 304, and dose dispensing mechanism 306. Based on the collected information, hardware processor 309 may control the actuators of sensor(s) and actuator(s) 308 to change the operations of dose control mechanism 304 and/or dose dispensing mechanism 306. For example, as to be described in more details below, the sensors of sensor(s) and actuator(s) 308 may generate data related to a movement of the piston shaft or knob of dose control mechanism 304. Based on the data, hardware processor 309 may determine a net movement of the piston shaft or knob, and a dosage set by a user based on the net movement. Hardware processor 309 may compare the dosage against a threshold to determine whether a correct dosage has been set. Hardware processor 309 may also compare the dosage against a set of discrete dosage levels to find the closest discrete dosage level. If hardware processor 309 determines that the dosage is incorrect, hardware processor 309 may control the actuator 308 of sensor(s) and actuator(s) 308 to disable dose dispensing mechanism 306, to prevent medicine delivery device 110 from administering an incorrect dosage of the medicine to the user. In some embodiments, hardware processor 309 may also set the dosage to the closest discrete dosage level (e.g., by controlling does control mechanism 304) to correct for the dosage error and to administer a dosage of the medicine according to the discrete dosage level.

Moreover, medicine delivery device 110 may include a communication interface 310 and an output interface 312. Communication interface 310 may communicate using wireless and/or wired means (e.g., via wireless communication link 120 and/or 125 of FIG. 1). Communication interface 310 may enable transmission of information related to administering the drug. For example, communication interface 310 may enable transmission of information indicating a dosage set by the user and, in the event that an incorrect dosage is set, may enable transmission of a warning to the user about the incorrect dosage. In some embodiments, communication interface 310 may enable transmission of information to a third party (e.g., a drug adherence or compliance system) for enforcing one or more pre-determined drug adherence or compliance rules. The information may then be displayed via an interface to, for example, assist the user in administering of the medicine, to notify the user (or the third party) about potential over dosage, etc. Moreover, as part of an interactive process, communication interface 310 may also receive information related to a confirmation (or an overriding command) from the user that the medicine is to be administered according to the set dosage. Communication interface 310 may relay the confirmation or overriding command to sensor(s) and actuator(s) 308, to enable dose dispensing mechanism 306 to dispense the medicine.

On the other hand, output interface 312 may be controlled by hardware processor 309 to output operation information of medicine delivery device 110. For example, output interface 312 may output (e.g., in display form, in audio form, etc.) a dosage set by the user. The output dosage information may be adjusted based on the configuration of dose control mechanism 304, to accounting for volumetric changes of the medicine and of medicine cartridge 302 as well as dose control mechanism 304. The user may use the output dosage information as a guide to set the dosage. Output interface 312 may include, for example, a display interface and/or an audio device (e.g., to display or speak out a set dosage), a mechanical device (e.g., to produce a click sound whenever a pre-determined unit dosage is selected and is added or subtracted from an initial dosage), etc.

Although not shown in FIG. 3, medicine delivery device 110 may further include one or more non-transitory storage devices including, for example, a solid-state storage device, such as a random access memory (“RAM”), and/or a read-only memory (“ROM”), which may be programmable, flash-updateable and/or the like. Such storage devices may be configured to implement any appropriate data stores, including without limitation, various file systems, database structures, and/or the like. A set of these instructions and/or code might be stored on a non-transitory computer-readable storage medium, which may then be executed by hardware processor 309 to perform the operations described above and operations as to be described below. The storage device may also store other data including, for example, threshold information, discrete dosage levels, sensor data, etc., to facilitate the operation of medicine delivery device 110.

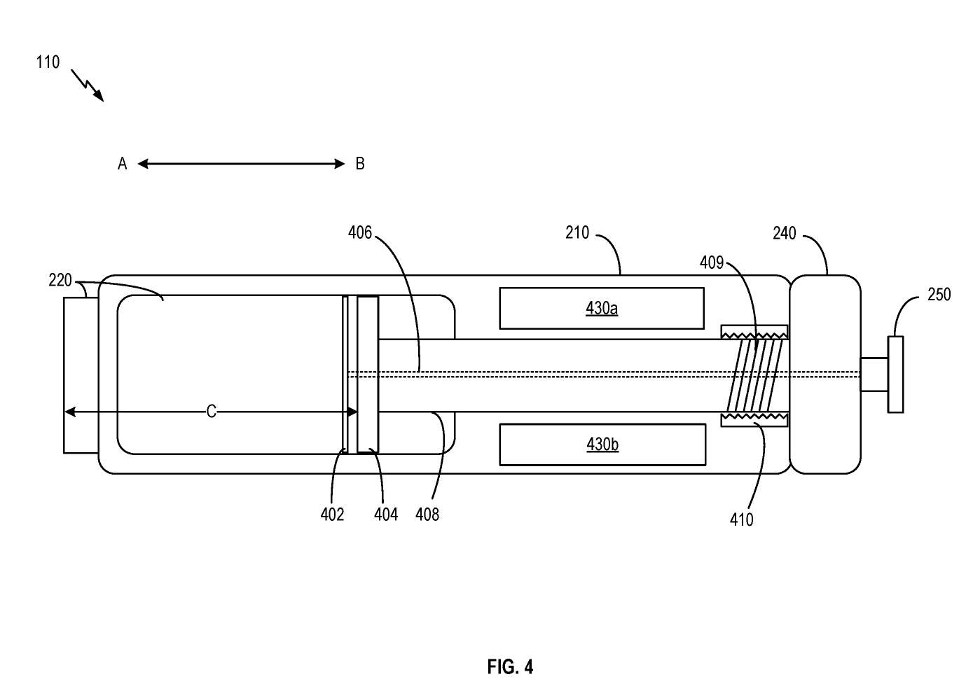

FIG. 4 is a simplified illustration of an internal structure of medicine delivery device 110, according to an example. It will be appreciated by a person of ordinary skill in the art that the illustration is not to scale, and the various components illustrated may vary in size, shape, arrangement, and/or other was, as desired. As shown in FIG. 4, medicine delivery device 110 may include a dispensing piston 402 and a dose setting piston 404 which may correspond to, respectively, piston 260 and head 265 of FIG. 2B. Dispensing piston 402 is coupled with dose dispensing button 250 via a shaft 406, whereas dose setting piston 404 is coupled with dose knob 240 via a screw shaft 408. Screw shaft 408 may include a set of helical grooves 409 coupled with body 210 via screw receiver 410. As screw shaft 408 rotates and guided by helical grooves 409 and screw receiver 410, screw shaft 408 may also perform a linear motion. For example, as a user rotates dose knob 240 in one direction (e.g., a clockwise direction or a counter-clockwise direction), screw shaft 408 may push dose setting piston 404 towards direction A, or pull dose setting piston 404 towards direction B. The distance between dose setting piston 304 and the left edge of reservoir chamber 220 (indicated by C) may define the volume of the medicine to be dispensed, which may correspond to the dosage selected by the user. After the user finishes rotating dose knob 240 and selects the dosage, the user may push down dose dispensing button 250, which then pushes dispensing piston 402 towards direction A to push the medicine out of reservoir chamber 220. In some embodiments, dose dispensing piston 402, dose setting piston 404, shaft 406, screw shaft 408, and dose dispensing button 250 may be part of a plunger. Dose setting piston 404, screw shaft 408, and dose knob 240 may be part of dose control mechanism 304 of FIG. 3, whereas dose dispensing piston 402, shaft 406, and dose dispensing button 250 may be part of dose dispensing mechanism 306. Dose knob 240, screw shaft 408, helical grooves 409, and screw receiver 410 may correspond to (or have similar functions as) dose selector 285 and nut 280 of FIG. 2B.

In addition, body 210 also holds electronic units 430a and 430b. Electronic units 430a and 430b may include sensors, actuators, and processors of sensor(s) and actuator(s) 308 to detect a dosage set by the user based on a net movement of dose setting piston 404 towards direction A. In some embodiments, electronic units 430a and 430b may include actuators to lock dispensing piston 402 (and/or shaft 406) at a fixed position to prevent dispensing piston 402 from pushing the medicine out of reservoir chamber 220, in the event that an incorrect dosage is detected. In some embodiments, electronic units 430a and 430b may also include actuators to rotate shaft 406 to adjust a dosage to reduce dosage error. Moreover, electronic units 430a and 430b may include communication interface circuitries of communication interface 310. The communication interface circuitries may transmit information about the dosage set, and receive a confirmation or overriding command to release the locking of dispensing piston 402 (and/or shaft 406), as described above.

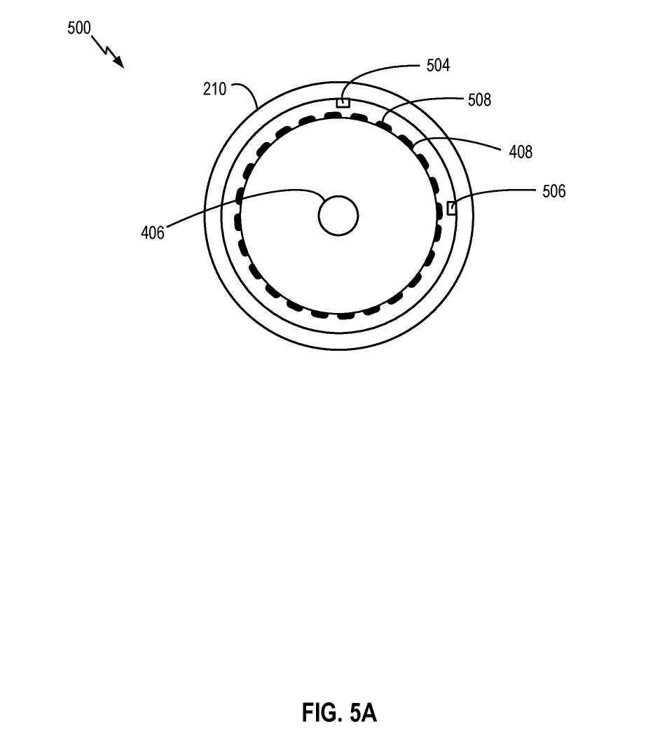

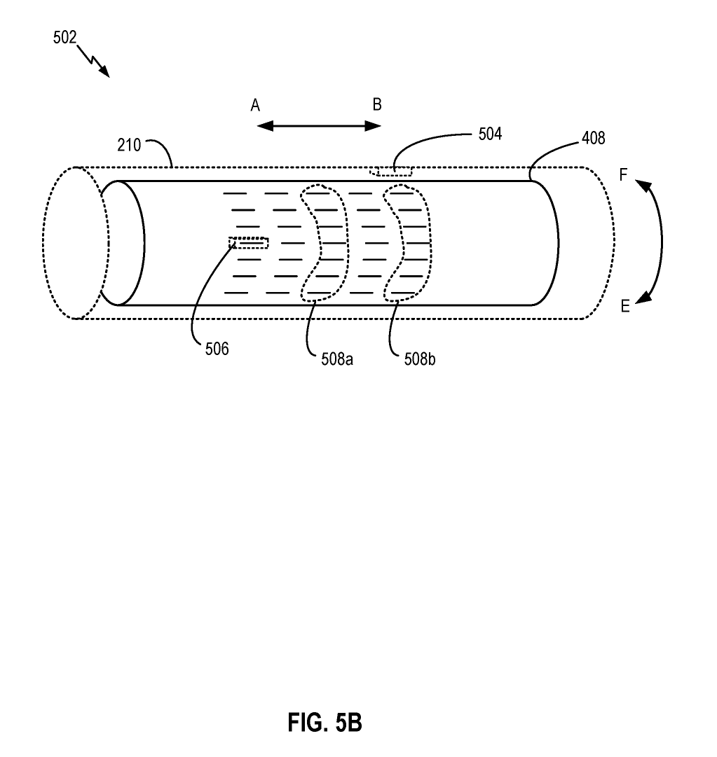

FIGS. 5A and 5B provide illustrations of embodiments of components of medicine delivery device 110 for sensing a movement of dose setting piston 404 for dosage estimation, according to certain aspects of the present disclosure. FIG. 5A illustrates an example of a cross section diagram 500 of body 210, shaft 406, and screw shaft 408 of medicine delivery device 110, whereas FIG. 5B illustrates a corresponding perspective diagram 502. As discussed above with reference to FIG. 4, dose setting piston 404 may be coupled with a screw shaft 408, which in turn is coupled with dose knob 240. As a user turns dose knob 240, screw shaft 408 may rotate while moving linearly along directions A or B. The linear movement of screw shaft 408 may push or pull dose setting piston 404. Therefore, the distance and direction of movement of dose setting piston 404 may be tracked by determining the distance and direction of movement of screw shaft 408.

Referring to FIG. 5A and FIG. 5B, medicine delivery device 110 includes two sensors 504 and 506 disposed with an inner surface of body 210. Sensors 504 and 506 may be part of sensor(s) and actuator(s) 308 of FIG. 3 and part of electronic units 430a and 430b of FIG. 4. As screw shaft 408 rotates, sensors 504 and 506 may sense a set of discrete detection targets 508 on the outer surface of screw shaft 408, and generate a time-series of data samples representing the detection (or non-detection) of a discrete detection target at different times. Processor 309 can determine a direction and a distance of movement of screw shaft 408 (e.g., along the A-B axis of FIG. 4) based on the time-series data samples. In some embodiments, processor 309 can determine the direction and the distance based on raw data samples from sensors 504 and 506. In some embodiments, processor 309 can perform post-processing (e.g., filtering) of the raw data samples and perform the direction and distance determination based on the post-processed data.

Referring back to FIG. 4, the set of discrete detection targets 508 may be located at a different location of screw shaft 408 from where the set of helical grooves 409 is located. For example, the set of discrete detection targets 508 may be in the region between electronic units 430a and 430b. The set of detection targets 508 may be divided into different groups comprising, for example, detection target groups 508a and 508b. Each detection target group includes a plurality of detection targets evenly distributed along a circumference of outer surface of screw shaft 408. Each detection target within a detection target group may correspond to a unit of an angular position of screw shaft 408. On the other hand, each detection target group may correspond to a unit of a linear position of screw shaft 408. By tracking a total number of detection targets that pass through a particular location within body 210, a net movement of screw shaft 408 within body 210 may be determined. For example, assuming each detection group has N detection targets, a net linear distance of movement by screw shaft 408 may be determined from the total count of detection targets based on the following equation:

$$Linear distance = \frac{Total}{N} \times L \qquad ( Equation 1 )$$

In Equation 1, the linear distance may be a net distance moved by screw shaft 408 towards direction A of FIG. 5. Total may be the total count of detection targets towards direction A (or direction B). N may be a number of detection targets within the detection target groups that have moved through sensors 504 and 506 as screw shaft 408 rotates and moves linearly towards direction A or direction B.

The total count of detection targets may be determined based on sensing data generated by sensors 504 and 506. For example, as screw shaft 408 rotates and move towards directions A or B, sensors 504 and 506 may detect the presence or the absence of a detection target, and the detection of a detection target may trigger a counter to update a count value. The count value may represent a number of detection targets (either from the same detection target group or from neighboring detection target groups) passing by. Specifically, sensors 504 and 506 may generate data of a pattern of absence and presence of a detection target that corresponds to the passing-by detection targets. As to be described in more details below, based on the patterns of absence and presence of the detection target provided by sensors 504 and 506, a processing circuit (e.g., hardware processor 309) may determine whether or not to update a counter that tracks the number of detection targets passing through one of sensors 504 or 506. Moreover, the processor may also determine a direction of movement of screw shaft 408 (e.g., whether it moves towards directions A or B), and to increment or decrement the counter based on the determined direction, to determine the net movement of screw shaft 408.

Various techniques may be used to perform the sensing of the detection targets. In some embodiments, each of the set of detection targets 508 may include a protrusion structure, and sensors 504 and 506 may each include a mechanical switch. As screw shaft 408 moves, one of the mechanical switches may be turned on when it comes in contact with a protrusion structure. The mechanical switch may then be turned off when the protrusion structure moves away, and may be turned on again when the next protrusion structure comes in contact. The turning-on of the mechanical switch may cause the sensor (e.g., one of sensors 504 and 506) to generate a voltage/current signal corresponding to the presence of a detection target, which may then be captured by the processor for direction and distance determination.

In some embodiments, sensors 504 and 506 may include optical sensors (e.g., visible light sensors, infra-red light sensors, ultra-violet light sensors, etc.), whereas each of the set of detection targets 508 may also include a marking of which an image may be captured by the optical sensors. For example, medicine delivery device 110 may include a light source within body 210 to project light (e.g., visible light, infra-red light, ultra-violet light, etc.) upon the outer surface of screw shaft 408. As screw shaft 408 moves, a marking may reflect the light towards one of sensors 504 and 506 at one time point and then deflect the light away from the sensors at another time point. Upon detection of the reflected light, the sensor may generate a voltage/current signal corresponding to the detection, which may then be captured by the processor for direction and distance determination.

In some embodiments, sensors 504 and 506 may also be coupled with an electronic switch (e.g., a transistor), whereas each of the set of detection targets 508 may include a capacitor that stores charges and become a voltage source. As screw shaft 408 moves, the gate terminal of the electronic switch may come in contact with a capacitor corresponding to one of the detection targets, and may be turned on. The electronic switch may then be turned off when the capacitor moves away, and may be turned on again when the next capacitor (corresponding to the next detection target) comes in contact. The turning-on of the electronic switch may also cause the sensor (e.g., one of sensors 504 and 506) to generate a voltage/current signal corresponding to the presence of a detection target, which may also be captured by the processor for direction and distance determination.

Although FIG. 5A and FIG. 5B show that two sensors (sensors 504 and 506) are used to estimate a net movement of screw shaft 408, it is understood that a single sensor may also be used to achieve the same result. For example, each of the set of detection objects may include a unique reflective marking representing, for example, a number, a symbol, etc. As screw shaft 408 moves, the sensor may output data corresponding to a sequence of appearances of the reflective markings. A processor may recognize the reflective markings and store a sequence of numbers or symbols it has recognized. The processor may determine the direction of movement of screw shaft based on a specific sequence of numbers of symbols detected. The processing circuit may also determine a distance moved by the screw shaft based on, for example, a number of numbers or symbols the processor has received. The processing circuit may also reset the stored sequence (and the determined direction and distance of movement of the movable component) based on a user input. The user input may be, for example, a reset signal, a set of inputs (e.g., from dose knob 240) to rotate screw shaft 408 back to its initial position, etc.

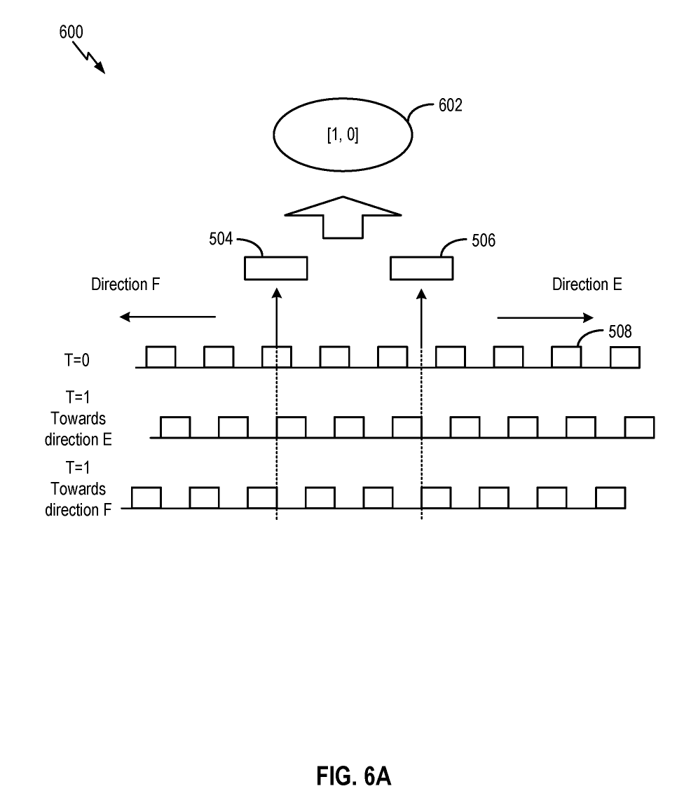

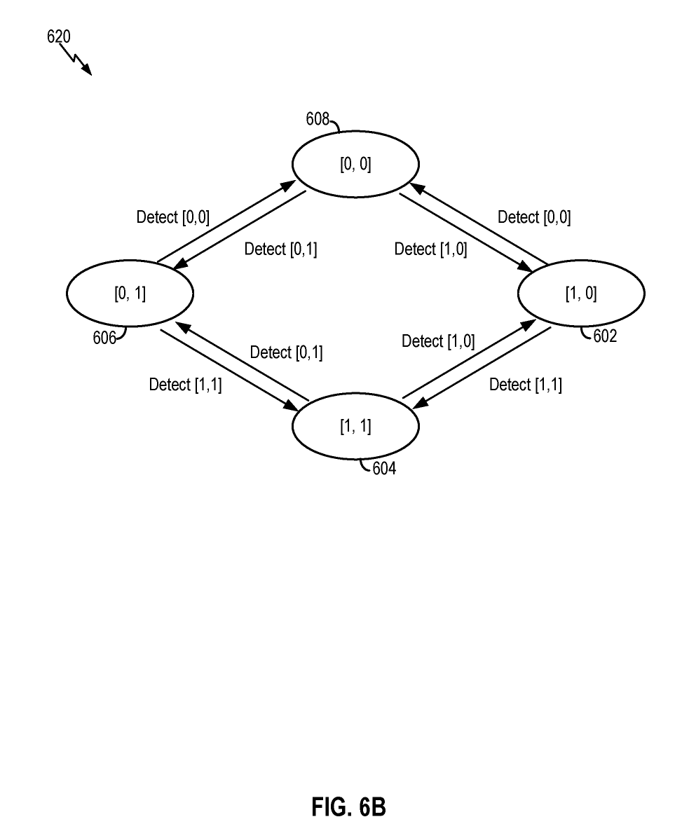

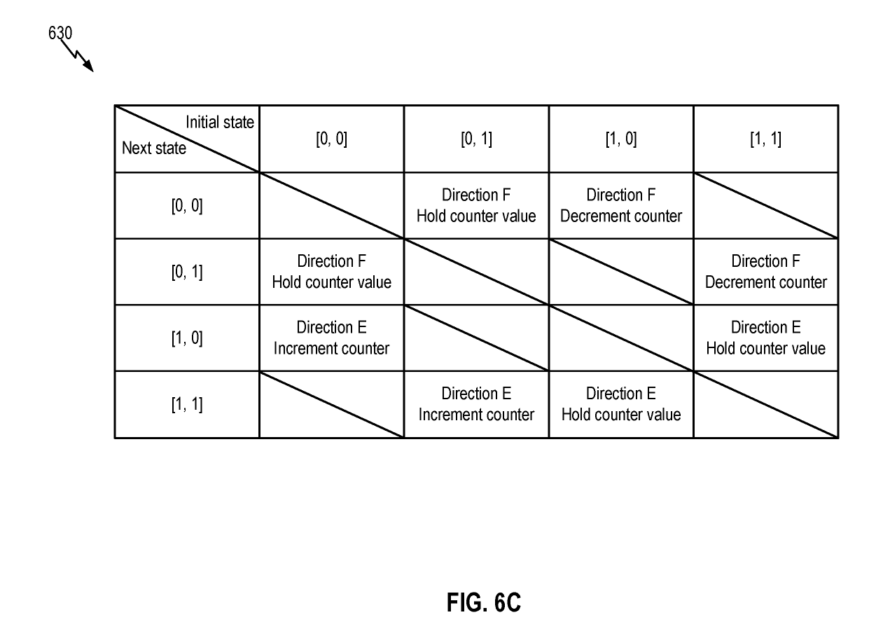

FIG. 6A, FIG. 6B, and FIG. 6C provide illustrations of operations for determining a direction of movement and updating a counter based on data generated by sensors 504 and 506 for dosage estimation, according to certain aspects of the present disclosure. Diagram 600 of FIG. 6A illustrates a simplified view of sensors 504 and 506 as well as some of the set of detection targets 508, whereas diagram 620 of FIG. 6B illustrates a set of possible internal states maintained by the processor based on the data generated by sensors 504 and 506. At shown in diagram 600, sensor 504 detects a presence of a detection target (e.g., presence of a protrusion structure, a capacitor, a reflective marking, etc.), whereas sensor 506 detects an absence of the detection target. Based on the output of sensors 504 and 506, a processing circuit (e.g., hardware processor 309) that controls the counter may set an internal state 602 of “[1,0]”, with the first entry (logical one) representing the presence detection result from sensor 504, and the second entry (logical zero) representing the absence detection result from sensor 506. Referring to diagram 620, in addition to internal state 602, the processor may also maintain, at different time points, internal state 604 (“[1, 1]”, representing a presence detection result from both sensors 504 and 506), internal state 606 (“[0, 1]”, representing an absence detection result from sensor 504 and a presence detection result from sensor 506), and internal state 608 (“[0,0]”, representing absence detection result from both sensors 504 and 506). Diagram 620 also shows the conditions for the transitions between the internal states. For example, referring to FIG. 6A, assuming that internal state 602 is the state at time t=0, if screw shaft 408 (and the set of detection targets 508) rotates towards direction E at time t=1, both sensors 504 and 506 may detect the presence of a detection target, and the next internal state may be internal state 604 (“[1,1]”). On the other hand, if at time t=1 screw shaft 408 (and the set of detection targets 508) rotates towards direction F, and the next internal state may be internal state 608 (“[0,0]”).

Accordingly, based on the transitions between the internal states, the processor may determine a direction of movement of screw shaft 408. FIG. 6C illustrates a table 630 that shows the direction of movement of the set of detection targets 508 that would have caused a particular transition between two internal states. For example, referring to table 630, based on a transition from an initial internal state of “[0,0]” to a next internal state of “[0,1]” (i.e., transition from internal state 608 to internal state 606, the processor may determine that the set of detection targets 508 (and screw shaft 408) rotates towards direction E. The processor may also determine the directions of rotation based on other internal state transitions as shown in table 630. Table 630 may be stored in a memory accessible by the processor.

Moreover, the processor may also update the counter based on detection of change of detection result in one of the sensors, to count a number of detection targets that have moved through that sensor. Referring to table 630, as an illustrative example, the processor may be configured to update the counter whenever it detects a change in the detection result of sensor 504. Based on this configuration, the processor may update the counter when there is a transition between internal states 608 and 602, or when there is a transition between internal state 606 and 604. The processor may increment or decrement the counter based on the direction of rotation. For example, assuming that when rotating in direction E screw shaft 408 moves towards reservoir chamber 220 and reduces the dosage, the processor may decrement the counter when there is a transition from internal state 608 to internal state 602, which corresponds to a movement in direction E. Likewise, when there is a transition from internal state 602 to internal state 608, which corresponds to a movement in direction F, the processor may increment the counter.

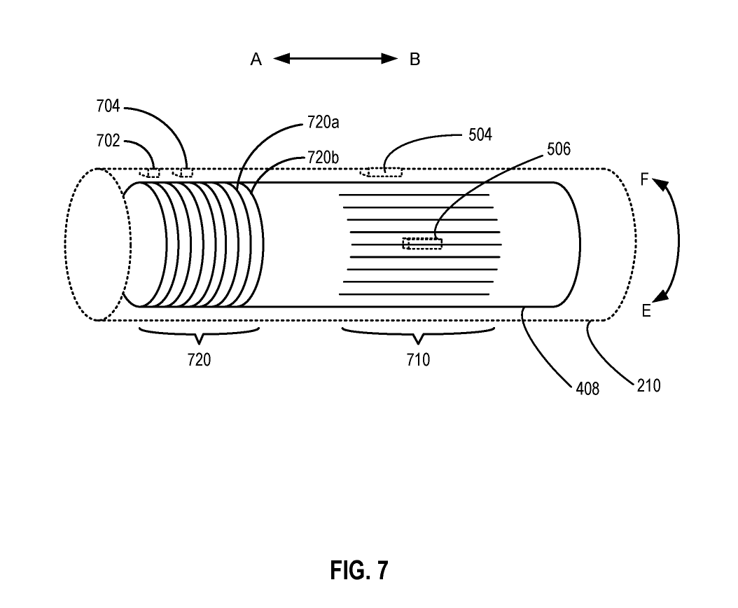

FIG. 7 provide illustrations of embodiments of components of medicine delivery device 110 for determining a direction and a distance of movement of screw shaft 408 for dosage estimation, according to certain aspects of the present disclosure. As shown in FIG. 7, in addition to sensors 504 and 506, medicine delivery device 110 may also include sensors 702 and 704. Sensors 504 and 506 may be configured to measure an angular position of screw shaft 408 based on a set of first detection targets 710, whereas sensors 702 and 704 may be configured to measure a linear position of screw shaft 408 based on a set of second detection targets 720. Both detection targets 710 and 720 may be located on at a different location of screw shaft 408 from where the set of helical grooves 409 is located. For example, detection targets 710 and 720 may be located in the region between electronic units 430a and 430b. Similar techniques disclosed with respect to FIGS. 5 and 6 may be used to perform the sensing of detection targets as well as distance and direction determination. For example, both first detection targets 710 and second detection targets 720 may include protrusion structures, capacitors, reflective markings, etc. Based on the sensors' detection results, a processor may count a number of detection targets passing through one of sensors 702 or 704 to estimate a linear distance (e.g., towards directions A or B) of movement by screw shaft 408. The processor may also count a number of detection targets passing through one of sensors 504 or 506 to estimate an angular displacement of movement by screw shaft 408. The processor may maintain a first counter to count a number of detection targets passing through one of sensors 702 or 704, and a second counter to count a number of detection targets passing through one of sensors 504 or 506. The processor may increment or decrement the first counter and the second counter based on, respectively, a direction of the linear movement and a direction of the rotational movement, based on techniques disclosed in FIG. 6.

One potential advantage of the arrangements of FIG. 7 is that a reduced number of detection targets (as well as counting) is needed to achieve a particular decision. For example, two detection targets 720a and 720b of the set of second detection targets 720 may be used to represent a distance of a linear movement by screw shaft 408 after it completes one revolution of rotation movement. The angular position of screw shaft 408 within one revolution may be represented by the set of first detection targets 710, and may be used to represent a fraction of a distance of the linear movement by screw shaft 408. The same set of first detection targets 710 may be used to measure the angular position of screw shaft 408 regardless of the linear position of screw shaft 408. Because of the reduced number of detection targets, the chance of the detection targets being misplaced and not representing units of position measurements may be reduced. As a result, the count may more accurately represent the net movement of screw shaft 408 as well as the dosage.

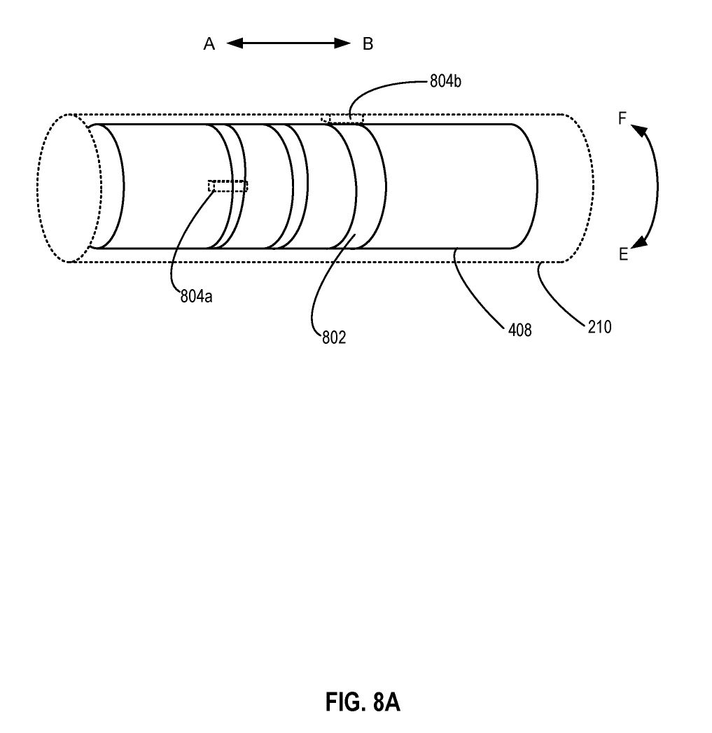

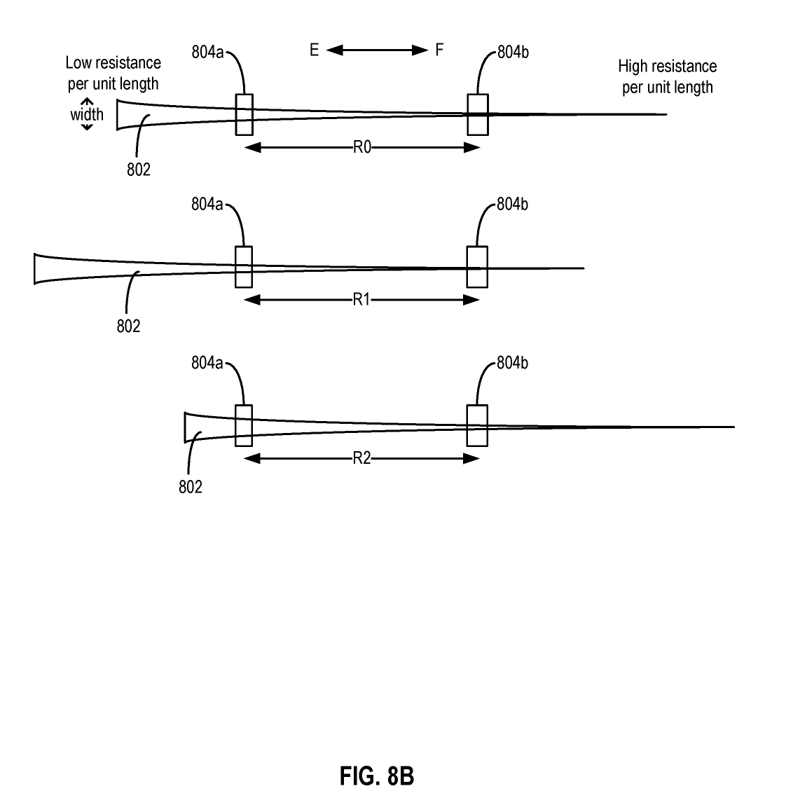

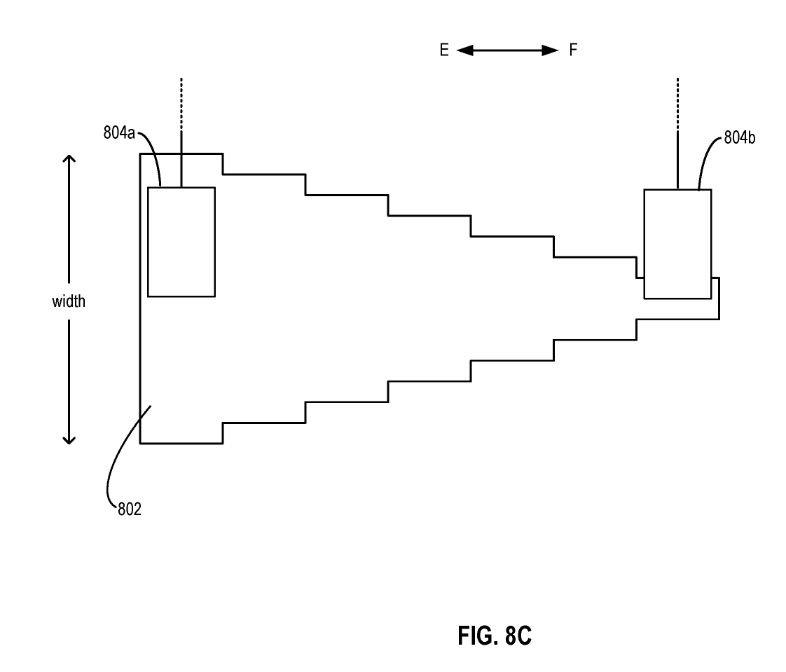

FIG. 8A provide illustrations of another embodiments of components of medicine delivery device 110 for determining a direction and a distance of movement of screw shaft 408 for dosage estimation, according to certain aspects of the present disclosure. As shown in FIG. 8, screw shaft 408 may include a resistive strip 802 wrapped around screw shaft 408 to form a helix. Resistive strip 802 may wrap around screw shaft 408 at a location different from where the set of helical grooves 409 is located. For example, resistive strip 802 may wrap around screw shaft 408 in the region between electronic units 430a and 430b. Medicine delivery device 110 may further include a pair of electrical contacts 804a and 804b that are electrically connected with resistive strip 802, such that a resistive path is formed between sensors 504 and 506 via a portion of resistive strip 802. Electrical contacts 804a and 804b may provide a pre-determined current through the portion of resistive strip 802 and develop a voltage difference. The voltage difference may be sensed by a sensor (e.g., a voltage sensor) coupled with for example, an analog-to-digital converter, which may convert the voltage into digital data and provide the digital data to a processing circuit (e.g., hardware processor 309). Hardware processor 309 may then determine the resistance and, based on the change in the resistance, estimate a direction and a distance of movement by screw shaft 408.

FIG. 8B illustrates additional details of resistive strip 802. For example, as shown in FIG. 8B, resistive strip 802 may be configured to have uneven resistance distribution, such that the resistance of the resistive path formed sensors 504 and 506 may vary based on a position of resistive strip 802 with respect to sensors 504 and 506. In the example of FIG. 8B, resistive strip 802 may have non-uniform width at different locations along the strip, which leads to different unit resistances (e.g., measured with respect to a unit length along E-F direction) at each of these locations. As a result, the resistance of the resistive path between sensors 504 and 506 may change when resistive strip 802 (and screw shaft 408) moves with respect to the sensors. Assuming at a certain point the resistance between sensors 504 and 506 is R0. If resistive path 802 moves towards direction E, the strip between sensors 504 and 506 becomes narrower, and the resistance between sensors 504 and 506, which becomes R1, may be larger than R0. On the other hand, if resistive path 802 moves towards direction F, the strip between sensors 504 and 506 becomes wider, and the resistance between sensors 504 and 506, which becomes R2, may be smaller than R0.

In some embodiments, instead of having a smooth change in the width as shown in FIG. 8B, resistive strip 802 may also have step changes in the width, as shown in FIG. 8C. The step changes may facilitate detection of small changes in the resistance, and improve the precision for determining the physical distance change.

The processor may determine a direction of movement of resistive strip 802 (and screw shaft 408), as well as the distance of the movement, by tracking the changes in the resistance between sensors 504 and 506. For example, as explained above, the processor may determine a direction of movement by resistive strip 802 based on whether the resistance increases or decreases. Moreover, the change in the resistance may also reflect the distance moved by resistive strip 802. For example, a mapping table may be used to store a set of resistances mapped to a set of positions of resistive strip 802. As resistive strip 802 moves, the processor may refer to the mapping table to track a position of the strip based on the resistance values, and may determine a net movement of the strip (and screw shaft 408) by tracking the changes in the position.

Based on the determined distance of movement as well as the direction of the movement, hardware processor 309 may estimate a dosage being set by tracking the changes in the position of dose setting piston 404. For example, referring back to FIG. 4, hardware processor 309 may accumulate the distances of movements of dose setting piston 404, while accounting for the directions of the movements, to find a net displacement of dose setting piston 404. The net displacement may be used to determine distance C which, combined with the cross-sectional area of reservoir chamber 220, may provide an estimate of an actual dosage.

Based on the actual dosage, processor 309 may be perform one more actions. For example, if the actual dosage exceeds a threshold, which may indicate dosage error, over dosage, etc., processor 309 may control the actuators to lock dispensing piston 402 (and/or shaft 406) at a fixed position to prevent dispensing piston 402 from pushing the medicine out of reservoir chamber 220. Processor 309 may also control the actuators to rotate screw shaft 408 (while guided by the sensor data from the sensors) to adjust the actual dosage to match the closest pre-determined discrete dosage level to reduce or eliminate the dosage error. Processor 309 may also control communication interface 310 to transmit a notification to the user, or to a third party, that an incorrect dosage is set and/or over dosage is likely. Processor 309 may also receive an overriding command (e.g., from the user, from another third party, etc.) over communication interface 310 to administer the dosage as set by the user without further adjustment, and processor 309 may release the lock on dispensing piston 402 (and/or shaft 406) to allow the administering of the dosage of the medicine. After the medicine has been dispensed, processor 309 may also erase the pattern of detection targets and/or physical quantity measurements from the memory, to prepare for the next measurement of distances and directions of movement of dose control mechanism 304. Processor 309 may also determine whether there is insufficient dosage left in reservoir chamber 220 and may lock dispensing piston 402 (and/or shaft 406) and/or transmit or display a notification to the user about the insufficient dosage.

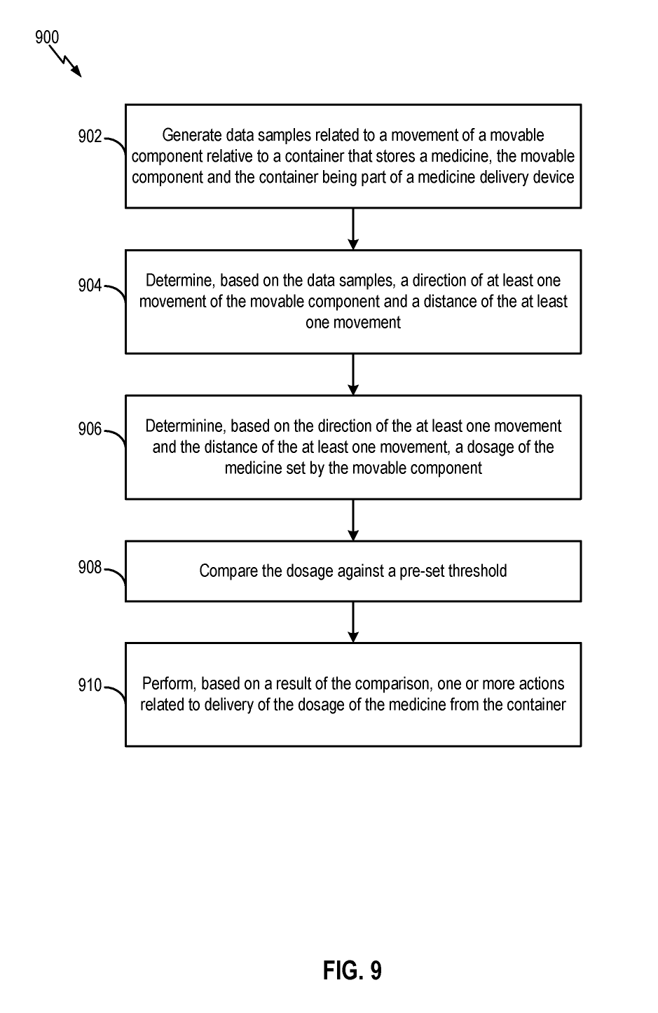

FIG. 9 includes a flowchart 900 which illustrates an example method for facilitating administering of medicine according to certain aspects of the present disclosure. The process illustrated by flowchart 900 may be performed by various components of medicine delivery device 110 described in FIG. 5A-FIG. 8C including, for example, sensors 504 and 506, sensors 702 and 704, electrical contacts 804a and 804b, and hardware processor 309.

At block 902, the system may generate data samples related to a movement of a movable component relative to a container that stores a medicine, the movable component and the container being part of a medicine delivery device. The data samples can include, for example, an indication of presence or an indication of absence of a detection target (e.g., as shown in FIG. 5A, FIG. 5B, and FIG. 7), a resistance measurement (e.g., as shown in FIG. 8B and FIG. 8C), etc. Means for performing the functionality of block 902 may include, for example, sensors 504 and 506, sensors 702 and 704, electrical contacts 804a and 804b, hardware processor 309, discrete detection targets 508, 710, and 720 on the outer surface of screw shaft 408, etc.

At block 904, the system may determine, based on the data samples, a direction of at least one movement of the movable component and a distance of the at least one movement. In some embodiments, referring back to FIG. 6B, the system can maintain an internal state representing a prior detection of indications of absence or presence of a detection target at two sensors (e.g., sensors 504 and 506). Based on the current internal state and the current indications of absence or presence of the detection target derived from the data samples of block 902, the system can determine the next internal state. Moreover, referring back to FIG. 6C, based on identifying a particular prior internal state and a particular current internal state transitioned to, the system can also determine a direction of movement, and whether to increment or decrement a counter that reflects a distance of movement of screw shaft 408 (and dose setting piston 404). In some embodiments, referring to FIGS. 8A to 8C, the system can also track a change in the resistance between electrical contacts 804a and 804b and, based on the change in resistance, determine a direction of movement and a distance of the movement of screw shaft 408. Means for performing the functionality of block 904 may include, for example, hardware processor 309.

At block 906, the system may determine, based on the direction of the at least one movement and the distance of the at least one movement, a dosage of the medicine set by the movable component. For example, referring back to FIG. 4, the system can determine, based on the distance and direction of movement of screw shaft 408, a distance (e.g., distance C) between dose setting piston 404 and an edge of reservoir chamber 220, and estimate the dosage of the medicine set by screw shaft 408 and dose setting piston 404 based on the distance. Means for performing the functionality of block 906 may include, for example, hardware processor 309.

At block 908, the system may compare the dosage against a pre-set threshold. The pre-set threshold may include, for example, a maximum amount beyond which over-dosage of the medicine may occur. The pre-set threshold may also include, for example, a pre-set proper dosage level. For example, the medicine delivery device may be configured to deliver a discrete set of dosages (e.g., in the steps of 0.5 mL, 1 mL, 1.5 mL, etc.), and the dosage set by the movable component can be compared against the discrete set of dosage levels to determine whether the dosage matches any of the discrete set of dosage levels. Means for performing the functionality of block 908 may include, for example, hardware processor 309.

At block 910, the system may, based on a result of the comparison, one or more actions related to delivery of the dosage of the medicine from the container. In a case where the dosage matches the threshold, the system may allow the medicine to be dispensed. For example, the system may remove a lock on dispensing piston 402, to allow the medicine to be pushed out of reservoir chamber 220 by the movement of dispensing piston 402. After the medicine has been dispensed, the system may also reset the estimated dosage to zero. On the other hand, if the system determines that the dosage does not match the pre-set threshold, the system may perform other actions. For example, the system may lock dispensing piston 402 at a fixed position within reservoir chamber 220 to prevent the medicine from being pushed out. The system may also transmit a notification about dosage error to the user (e.g., to connecting device 130). The system may also determine the closest discrete dosage level, and adjust the actual dosage (e.g., by rotating screw shaft 408) to match with the closest discrete dosage level to reduce the dosage error due to precision limitation of human operation. The system may transmit (e.g., via communication interface 310) the dosage information to a drug adherence or a compliance system. The drug adherence or compliance system may or may not be part of stakeholder(s) 160 of FIG. 1. The drug adherence or compliance system may maintain a record of the administering of the dosage of the medicine. The drug adherence or compliance system may also determine whether the administering of the medicine complies with a set of pre-determined rules based on the record as well as other physical parameters (e.g., time, physical condition of the user, etc.). For example, the pre-determined rules may specify a minimum time gap between administering of two doses of a medicine. The drug adherence or compliance system may check the compliance of the pre-determined rules based on the dosage information received from the medicine delivery device and the times when the information is received. If the drug adherence or compliance system determines that the pre-determined rules are not being complied, the drug adherence or compliance system may perform other actions including, for example, notifying the authority, transmitting a signal to the medicine delivery device to lock the dispensing mechanism, etc. Means for performing the functionality of block 910 may include, for example, hardware processor 309, screw shaft 408, dose setting piston 404, dose dispensing mechanism 306 including dose dispensing button 250, communication interface 310, output interface 312, etc.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations may be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

1. A medicine delivery device, comprising:
a container configured to store a medicine;
a movable component coupled with the container and configured to set a dosage of the medicine to be dispensed;
a dispensing mechanism coupled with the container and configured to deliver the dosage of the medicine;
one or more sensors configured to generate data samples related to a movement of the movable component; and
a processing circuit coupled with the one or more sensors and configured to:
determine, based on the data samples, a direction of at least one movement and a distance of the at least one movement;
determine, based on the direction of the at least one movement and the distance of the at least one movement, a dosage of the medicine set by the movable component;
compare the dosage against a pre-set threshold; and
perform one or more actions related to delivery of the dosage of the medicine from the container based on a result of the comparison.

2. The medicine delivery device of claim 1, wherein the movable component includes one or more detection targets for the one or more sensors; and
wherein the data samples comprises an indication of absence and an indication of presence of a detection target of the one or more detection targets.

3. The medicine delivery device of claim 2, wherein the one or more sensors comprise at least two switches;
wherein the one or more detection targets comprise one or more structures capable of causing the at least two switches to enter an on-state or an off-state;
wherein the indication of absence of a detection target of the one or more detection targets comprises an indication that one of the at least two switches is in the off-state; and
wherein the indication of presence of a detection target of the one or more detection targets comprises an indication that the one of the at least two switches is in the on-state.

4. The medicine delivery device of claim 3, wherein the at least two switches are mechanical switches; and
wherein the one or more structures are capable of causing the at least two switches to enter the on-state by applying a force to the at least two switches.

5. The medicine delivery device of claim 3, wherein the at least two switches are transistor switches; and
wherein the one or more structures store charges and are capable of causing the at least two switches to enter the on-state by applying at least some of the charges to gates of the transistor switches.

6. The medicine delivery device of claim 2, wherein the one or more sensors comprise one or more optical sensors; and
wherein the one or more detection targets comprise one or more markers.

7. The medicine delivery device of claim 2, wherein the one or more detection targets include a resistive path between two contacts;
wherein the one or more sensors are coupled with the two contacts and are configured to generate data samples of a resistance measurement of the resistive path; and
wherein the processing circuit is configured to determine the direction of at least one movement and the distance of the at least one movement based on a change in the resistance measurement reflected in the data samples.

8. The medicine delivery device of claim 7, wherein the resistive path has an non-uniform distribution of resistance.

9. The medicine delivery device of claim 2, wherein the processing circuit is configured to:
determine a counter value based on the data samples;
determine a net displacement of the movable component based on the counter value; and
determine the dosage based on the net displacement.

10. The medicine delivery device of claim 9,
wherein the processing circuit is configured to:
determine, based on the data samples, whether the counter value is to be updated; and
based on a determination that the counter value is to be updated, increment or decrement the counter value based on whether the direction of the at least one movement is of a first direction or of a second direction.

11. The medicine delivery device of claim 10, wherein the determination of whether the counter value is to be updated comprises the processing circuit being configured to:
determine whether there is a change in the indication of absence or in the indication of presence of a detection target of the one or more detection targets between a current data sample and a prior data sample obtained by one of the one or more sensors; and
update the counter value based on the change.

12. The medicine delivery device of claim 10, wherein the one or more sensors comprises a first sensor and a second sensor; wherein the data samples comprise:
first data samples including a first sequence of indications of absence and indications of presence of a detection target obtained by the first sensor; and
second data samples including a second sequence of indications of absence and indications of presence of the detection target obtained by the second sensor; and
wherein the processing circuit is configured to determine the direction of the at least one movement based on the first data samples and the second data samples.

13. The medicine delivery device of claim 1, wherein the at least one movement comprises a linear movement and a rotational movement;
wherein the one or more sensors comprises:
a first set of one or more sensors configured to generate first data associated with the linear movement, and
a second set of one or more sensors configured to generate second data including an angular displacement of the rotational movement; and
wherein the processing circuit is configured to:
determine a combined distance of the linear movement based on a combination of the first data and the second data; and
determine the dosage based on the combined distance.

14. The medicine delivery device of claim 13, wherein the movable component comprises a piston coupled with a threaded shaft;
wherein the first data is associated with a pre-determined scale for measuring the linear movement of the piston; and
wherein the angular displacement included in the second data corresponds to a fraction of the pre-determined scale for measuring the rotational movement of the threaded shaft.

15. The medicine delivery device of claim 1, further comprising a wireless interface coupled with the processing circuit;
wherein the processing circuit is further configured to perform the one or more actions comprising:
transmitting information related to the delivery of the dosage of the medicine via the wireless interface to a client device associated with a user, to cause the client device to display the information;
transmitting the information for displaying at a display interface of the medicine delivery device;
or any combination thereof.

16. The medicine delivery device of claim 15, wherein the information comprise a notification of insufficient dosage of medicine left for dispensing.

17. The medicine delivery device of claim 15, wherein the information comprise an indication about the dosage.

18. The medicine delivery device of claim 15, wherein the information is transmitted to a drug adherence or compliance system for enforcing one or more pre-determined drug adherence or compliance rules.

19. The medicine delivery device of claim 1, wherein the processing circuit is further configured to perform the one or more actions comprising:
based on a determination that the dosage is not equal to the pre-set threshold, controlling the dispensing mechanism not to dispense the medicine, wherein the pre-set threshold comprises one of: a pre-set maximum amount, or a pre-set dosage.

20. The medicine delivery device of claim 1, wherein the processing circuit is further configured to perform the one or more actions comprising:
based on a determination that the dosage is not equal to the pre-set threshold, adjusting the dosage using the movable component such that the dosage becomes equal to the pre-set threshold.

21. A method comprising:
receiving, from a sensor, data samples related to a movement of a movable component relative to a container that stores a medicine, the sensor, the movable component and the container being part of a medicine delivery device;
determining, based on the data samples, a direction of at least one movement of the movable component and a distance of the at least one movement;
determining, based on the direction of the at least one movement and the distance of the at least one movement, a dosage of the medicine set by the movable component;
comparing the dosage against a pre-set threshold; and
controlling, based on a result of the comparison, the medicine delivery device to perform one or more actions related to delivery of the dosage of the medicine from the container.

22. The method of claim 21, wherein the movable component includes one or more detection targets for the one or more sensors; and
wherein the data samples comprises an indication of absence and an indication of presence of a detection target of the one or more detection targets.

23. The method of claim 22, wherein the one or more sensors comprise at least two switches;
wherein the one or more detection targets comprise one or more structures capable of causing the at least two switches to enter an on-state or an off-state;
wherein the indication of absence of a detection target of the one or more detection targets comprises an indication that one of the at least two switches is in the off-state; and
wherein the indication of presence of a detection target of the one or more detection targets comprises an indication that the one of the at least two switches is in the on-state.

24. The method of claim 23, wherein the at least two switches are mechanical switches; and
wherein the one or more structures are capable of causing the at least two switches to enter the on-state by applying a force to the at least two switches.

25. The method of claim 23, wherein the at least two switches are transistor switches; and
wherein the one or more structures store charges and are capable of causing the at least two switches to enter the on-state by applying at least some of the charges to gates of the transistor switches.

26. The method device of claim 22, wherein the one or more sensors comprise one or more optical sensors; and
wherein the one or more detection targets comprise one or more markers.

27. The method of claim 22, wherein the one or more detection targets include a resistive path between two contacts;
wherein the one or more sensors are coupled with the two contacts and are configured to generate data samples of a resistance measurement of the resistive path; and
wherein the method further determining the direction of at least one movement and the distance of the at least one movement based on a change in the resistance measurement reflected in the data samples.

28. The method of claim 27, wherein the resistive path has an non-uniform distribution of resistance.

29. The method of claim 22, further comprising:
determining a counter value based on the data samples;
determining a net displacement of the movable component based on the counter value; and
determining the dosage based on the net displacement.

30. The method of claim 29, further comprising:
determining, based on the data samples, whether the counter value is to be updated; and
based on a determination that the counter value is to be updated, incrementing or decrementing the counter value based on whether the direction of the at least one movement is of a first direction or of a second direction.

31. The method of claim 30, wherein determining whether the counter value is to be updated comprises:
determining whether there is a change in the indication of absence or in the indication of presence of a detection target of the one or more detection targets between a current data sample and a prior data sample obtained by one of the one or more sensors; and
updating the counter value based on the change.

32. The method of claim 30, wherein the one or more sensors comprises a first sensor and a second sensor; wherein the data samples comprise:
first data samples including a first sequence of indications of absence and indications of presence of a detection target obtained by the first sensor; and
second data samples including a second sequence of indications of absence and indications of presence of the detection target obtained by the second sensor; and
wherein the method further comprises determining the direction of the at least one movement based on the first data samples and the second data samples.

33. The method of claim 21, wherein the at least one movement comprises a linear movement and a rotational movement;
wherein the one or more sensors comprises:
a first set of one or more sensors configured to generate first data associated with the linear movement, and
a second set of one or more sensors configured to generate second data including an angular displacement of the rotational movement; and
wherein the method further comprises:
determining a combined distance of the linear movement based on a combination of the first data and the second data; and
determining the dosage based on the combined distance.

34. The method of claim 33, wherein the movable component comprises a piston coupled with a threaded shaft;
wherein the first data is associated with a pre-determined scale for measuring the linear movement of the piston; and
wherein the angular displacement included in the second data corresponds to a fraction of the pre-determined scale for measuring the rotational movement of the threaded shaft.

35. The method of claim 21, wherein performing the one or more actions comprises:
transmitting information related to the delivery of the dosage of the medicine via a wireless interface to a client device associated with a user, to cause the client device to display the information;
transmitting the information for displaying at a display interface of the medicine delivery device;
or any combination thereof.

36. The method of claim 35, wherein the information comprise a notification of insufficient dosage of medicine left for dispensing.

37. The method of claim 35, wherein the information comprise an indication about the dosage.

38. The method of claim 35, wherein the information is transmitted to a drug adherence or compliance system for enforcing one or more pre-determined drug adherence or compliance rules.

39. The method of claim 21, wherein performing the one or more actions comprising:
based on determining that the dosage is not equal to the pre-set threshold, controlling the dispensing mechanism not to dispense the medicine, wherein the pre-set threshold comprises one of: a pre-set maximum amount, or a pre-set dosage.

40. The method of claim 21, wherein performing the one or more actions comprising:
based on determining that the dosage is not equal to the pre-set threshold, adjusting the dosage using the movable component such that the dosage becomes equal to the pre-set threshold.

41. A non-transitory computer readable medium that stores a set of instructions that, when executed by a hardware processor, causes the hardware processor to perform:
receiving, from a sensor, data samples related to a movement of a movable component relative to a container that stores a medicine, the sensor, the movable component and the container being part of a medicine delivery device;
determining, based on the data samples, a direction of at least one movement of the movable component and a distance of the at least one movement;
determining, based on the direction of the at least one movement and the distance of the at least one movement, a dosage of the medicine set by the movable component;
comparing the dosage against a pre-set threshold; and
controlling, based on a result of the comparison, the medicine delivery device to perform one or more actions related to delivery of the dosage of the medicine from the container.

42. An apparatus, comprising:
means for storing a medicine;
means for setting a dosage of the medicine to be dispensed;
means for delivering the dosage of the medicine;
means for generating data samples related to a movement of the means for setting the dosage; and
means for determining, based on the data samples, a direction of at least one movement and a distance of the means for setting the dosage;
means for determining, based on the direction of the at least one movement and the distance of the at least one movement, a dosage of the medicine set by the means for setting the dosage;
means for comparing the dosage against a pre-set threshold; and
means for performing one or more actions related to delivery of the dosage of the medicine from the means for storing the medicine based on a result of the comparison.
